(19) United States
(12) Patent Application Publication
(10) Pub. No.: US 2009/0227552 A1
(43) Pub. Date: Sep. 10, 2009

(54) FULVESTRANT FORMULATIONS

(76) Inventors: Kellie Ann Hooley, Burwood (AU); Allan Harvey Spencer, Ferntree Gully (AU); Ash Daniel David, Hawthorn East (AU)

Correspondence Address: WINSTON & STRAWN LLP;PATENT DEPARTMENT, 1700 K STREET, N.W., WASHINGTON, DC, 20006, US

(21) Appl. No.: 12/088,150

(22) PCT Filed: Sep. 26, 2006
(86) PCT No.: PCT/AU06/01399

§ 371 Date: Jun. 23, 2008

(30) Foreign Application Priority Data
Sep. 26, 2005 (AU) 2005905299

(51) Int. Cl. A61K 31/565

(52) U.S. Cl. 514/182

(57) ABSTRACT

The present invention relates to novel fulvestrant formulations comprising one or more of a propylene glycol or a pharmaceutically acceptable polyethylene glycol.

### FIELD OF THE INVENTION

The invention relates to a novel formulation comprising fulvestrant.

### BACKGROUND OF THE INVENTION

Fulvestrant (7α-[9-(4,4,5,5,5-pentafluoropentylsuphinyl)nonyl]estra-1,3,5-(10)-triene-3,17-β-diol) is an estrogen receptor antagonist. The molecular structure of fulvestrant is shown by Formula I.

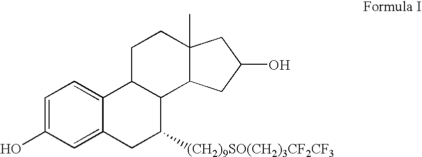

Fulvestrant is used in the treatment of hormone receptor positive breast cancer. Hormone receptor positive tumours are responsive to hormone levels, such as estrogen. As the growth of this type of tumour is dependent on the presence of hormones which stimulate tumour growth by binding to cellular hormone receptors, it is desirable to administer hormone receptor antagonists to block the growth signalling pathway. The administration of an estrogen receptor antagonist, such as fulvestrant, results in the down regulation of estrogen receptors in a hormone receptive positive tumour thereby preventing the effects of estrogen binding activity.

Fulvestrant formulations are administered intramuscularly. Intramuscular formulations need to have a high concentration of active as only relatively low volumes can be injected. Fulvestrant has a low solubility in many pharmaceutically acceptable solvents due to its lipophilicity and accordingly is difficult to formulate at appropriate concentrations. Formulations comprising castor oil and an alcohol, such as benzyl alcohol, are described in U.S. Pat. No. 5,183,814. However, it is stated in WO 01/51056 that the high proportion of alcohol required for this formulation may be undesirable for large scale manufacturing processes. In addition, it is well known that a high level of alcohol in a formulation for intramuscular injection increases the pain experienced by the subject after injection.

Formulations comprising castor oil together with an alcohol and a non-aqueous ester solvent are described in WO 01/51056. The alcohol and non-aqueous ester solvent are claimed to increase the overall solubility of the fulvestrant and to decrease the volume of alcohol utilised in the formulation so that the administration volume is therapeutically acceptable. A further advantage, according to WO 01/51056, is the reduction in the alcohol content, making for easier manufacturability. The preferred alcohols are ethanol and benzyl alcohol and the preferred non-aqueous ester solvents are benzyl benzoate, ethyl oleate, isopropyl myristate and isopropyl palmitate. The addition of the non-aqueous ester solvent is taught as being essential in achieving a fulvestrant formulation of at least 45 mg/mL and a total formulation volume of 6 mL or less. WO 01/51056 states that current guidelines recommend that no more than 5 mL of liquid should be injected intramuscularly in a single injection.

Surprisingly, the present inventors have found that propylene glycol or polyethylene glycol can be used in a novel fulvestrant formulation having sufficiently high concentration to be used intramuscularly thereby removing the need for a non-aqueous ester solvent. These novel formulations show stabilities suitable for pharmaceutical formulations and therefore provide alternative formulations for fulvestrant.

### SUMMARY OF THE INVENTION

In a first aspect, the present invention provides a formulation comprising an active compound selected from:
(i) fulvestrant or a pharmaceutically acceptable salt thereof; and
(ii) a pharmaceutically acceptable derivative of fulvestrant or a pharmaceutically acceptable salt thereof;
at least one pharmaceutically acceptable alcohol;

at least one of propylene glycol or a pharmaceutically acceptable polyethylene glycol;
and a castor oil carrier.

In a second aspect, the present invention provides a method for the treatment of hormone positive receptor tumours which comprises administering a formulation according to the first aspect of the invention to a patient in need thereof.

In a third aspect, the present invention provides a process for the preparation of a formulation according to the first aspect of the invention, comprising the steps of: (a) mixing the active compound with at least one alcohol; (b) adding at least one of propylene glycol or a polyethylene glycol; and (e) making up the required volume with castor oil.

In a fourth aspect, the present invention provides a formulation comprising:
(a) around 5% w/w of an active compound selected from:
   (i) fulvestrant or a pharmaceutically acceptable salt thereof; and
   (ii) a pharmaceutically acceptable derivative of fulvestrant or a pharmaceutically acceptable salt thereof;
(b) 10% w/w of ethanol;
(c) 10% w/w of benzyl alcohol;
(d) 15% w/w of PEG 300; and
(c) castor oil to make up the required weight.

### DETAILED DESCRIPTION OF THE INVENTION

In a first aspect, the present invention provides a formulation comprising an active compound selected from:
(i) fulvestrant or a pharmaceutically acceptable salt thereof; and
(ii) a pharmaceutically acceptable derivative of fulvestrant or a pharmaceutically acceptable salt thereof;
at least one pharmaceutically acceptable alcohol;
at least one of propylene glycol or a pharmaceutically acceptable polyethylene glycol;
and a castor oil carrier.

Preferably, the concentration of the effective amount of active compound is 2% w/w to 10% w/w. More preferably the concentration of the effective amount of active compound is 5% w/w.

By “effective amount of active compound”, we mean the proportion by mass of the active compound that is due to fulvestrant in circumstances where the active compound is a pharmaceutically acceptable derivative of fulvestrant, a pharmaceutically acceptable salt of fulvestrant or a pharmaceutically acceptable salt of a pharmaceutically acceptable derivative of fulvestrant.

Preferably, the pharmaceutically acceptable alcohol is selected from the group consisting of ethanol, benzyl alcohol or a mixture thereof. In a preferred embodiment, the concentration of ethanol is in the range 8% w/w to 15% w/w. More preferably the concentration of ethanol is 10% w/w. In yet another preferred embodiment the concentration of benzyl alcohol is in the range 5% w/w to 15% w/w. More preferably the concentration of benzyl alcohol is 10% w/w. More preferably the alcohol is a mixture of ethanol and benzyl alcohol. Preferably the concentration of the mixture of ethanol and benzyl alcohol is in the range 15% w/w to 25% w/w, more preferably the concentration is 20% w/w.

Preferably, the pharmaceutically acceptable polyethylene glycol is selected from the group consisting of PEG-200, PEG-300, PEG-400, and PEG-600. More preferably the polyethylene glycol is PEG-300. In a preferred embodiment, the concentration of the propylene glycol or polyethylene glycol is in the range 10% w/w to 20% w/w, more preferably the concentration is 15% w/w.

In a second aspect, the present invention provides a method for the treatment of hormone positive receptor tumours which comprises administering a pharmaceutically acceptable formulation according to the first aspect of the invention to a patient in need thereof.

Administration may be by any suitable means but is preferably by intramuscular injection.

Preferably, the hormone positive receptor tumour is breast cancer.

In a third aspect, the present invention provides a process for the preparation of a pharmaceutically acceptable formulation according to the first aspect of the invention, comprising the steps of: (a) mixing fulvestrant with at least one alcohol; (b) adding at least one or propylene glycol or a polyethylene glycol; and (c) making up the required volume with castor oil.

In a fourth aspect, the present invention provides a formulation comprising:
(a) around 5% w/w of an active compound selected from:
   (i) fulvestrant Or pharmaceutically acceptable salt thereof; and
   (ii) a pharmaceutically acceptable derivative of fulvestrant or a pharmaceutically acceptable salt thereof;
(b) 10% w/w of ethanol;
(c) 10% w/w of benzyl alcohol;
(d) 15% w/w of PEG 300; and
(e) castor oil to make up the required weight.

In order that the nature of the present invention may be more clearly understood, preferred forms thereof will now be described with reference to the following non-limiting examples.

### Example 1

### General Formulation Method

Tables 1 and 2 disclose formulations according to the present invention.

The general method of preparation of the formulations for a formulation having a total weight of 50 grams is as follows. Fulvestrant (2.5 g, 5% w/w) is mixed with ethanol and benzyl alcohol in the amounts defined for the particular formulation at a controlled rate and under shear. The polyol or polyethylene glycol in the amount defined for the particular formulation is then added following approximately 15 minutes of mixing of the alcohols and fulvestrant. This admixture is mixed for a further 15-20 minutes. The formulation is then made up to 50 g (100% w/w) with castor oil and the formulation mixed under shear.

The same general method would be used with any active compound encompassed by this specification.

TABLE 1
PEG-300 formulations

| | PEG-300 | Ethanol | Benzyl alcohol |
|---|---|---|---|
| Example No | (% w/w) | (% w/w) | (% w/w) |
| 1 | 15 | 8 | 12 |
| 2 | 15 | 9 | 11 |
| 3 | 15 | 10 | 10 |
| 4 | 15 | 11 | 9 |
| 5 | 15 | 12 | 8 |
| 6 | 15 | 15 | 5 |
| 7 | 13 | 10 | 12 |
| 8 | 13 | 11 | 11 |
| 9 | 13 | 12 | 10 |
| 10 | 10 | 15 | 10 |
| 11 | 10 | 12.5 | 12.5 |
| 12 | 10 | 10 | 15 |

TABLE 2
Propylene glycol formulations

| | Propylene glycol | Ethanol | Benzyl alcohol |
|---|---|---|---|
| Example No | (% w/w) | (% w/w) | (% w/w) |
| 1 | 15 | 8 | 12 |
| 2 | 15 | 9 | 11 |
| 3 | 15 | 10 | 10 |
| 4 | 15 | 11 | 9 |
| 5 | 15 | 12 | 8 |
| 6 | 15 | 15 | 5 |
| 7 | 13 | 10 | 12 |
| 8 | 13 | 11 | 11 |
| 9 | 13 | 12 | 10 |
| 10 | 10 | 15 | 10 |
| 11 | 10 | 12.5 | 12.5 |
| 12 | 10 | 10 | 15 |

### Example 2

### One Month Stability Results

The stability of the fulvestrant formulations of the present invention was determined at one month. The stability tests were conducted for each formulation at temperatures of 40° C., room temperature and 2-8° C. respectively. The percentage of fulvestrant was determined by HPLC analysis. The results are shown in Tables 3 and 4.

A comparison was also made with the innovator product Faslodex, the results of which are shown in Table 5. Faslodex is a controlled slow release formulation of fulvestrant for intramuscular injection. The formulation carrier is castor oil with excipients of benzyl alcohol, benzyl benzoate (a non-aqueous ester solvent) and ethanol.

The results demonstrate that there is no significant difference between the stability of the formulations of the prior art and those of the current invention.

### Example 3

### Stability Study After Four and a Half Months

All of the formulations (including the Faslodex formulations) tested in the one month stability study were also tested for stability after four and a half months. The formulations were all found to be extremely stable. In addition, the excipients were stable to visual inspection and no significant amount of benzaldehyde (a degradation product of benzyl alcohol) was detected in any of the formulations studied.

### HPLC Analysis

The HPLC analysis was carried out using the following parameters:

### Column: Alltech Platinum, Cyano, 5 μm, 4.6×250 mm

Column temperature: 50±2° C.
Sample temperature: 5±2° C.

### Detector Wavelength: 220 nm

Flow rate: 15 mL/min,
Injection volume: 10 μl,
Sample concentration: 5 mg/mL
API retention time: ˜23 min.
Total run time: 45 min.
Diluent: 30% (v/v) of n-propanol in n-hexane

TABLE 3
HPLC Results for PEG-300 formulations after 1 month stability trial

| | % Fulvestrant | % Fulvestrant | % Fulvestrant | % Fulvestrant |
|---|---|---|---|---|
| Formulation | at start date | at 40° C. | at Room Temperature | at 2-8° C. |
| PEG-300 (15%) | 99.67 | 99.44 | 99.64 | 99.57 |
| Ethanol (10%)/ | | | | |
| Benzyl alcohol | | | | |
| (10%) | | | | |
| PEG-300 (13%) | 99.67 | 99.45 | 99.63 | 99.59 |
| Ethanol (11%)/ | | | | |
| Benzyl alcohol | | | | |
| (11%) | | | | |
| PEG-300 (10%) | 99.68 | 99.43 | 99.64 | 99.59 |
| Ethanol (12.5%)/ | | | | |
| Benzyl alcohol | | | | |
| (12.5%) | | | | |

TABLE 4
HPLC Results for Propylene glycol formulations after 1 month stability trial

| | % Fulvestrant | % Fulvestrant | % Fulvestrant | % Fulvestrant |
|---|---|---|---|---|
| Formulation | at start date | at 40° C. | at Room Temperature | at 2-8° C. |
| Propylene | 99.65 | 99.62 | 99.65 | 99.59 |
| glycol (15%) | | | | |
| Ethanol (10%)/ | | | | |
| Benzyl alcohol | | | | |
| (10%) | | | | |
| Propylene | 99.64 | 99.60 | 99.64 | 99.59 |
| glycol (13%) | | | | |
| Ethanol (11%)/ | | | | |
| Benzyl alcohol | | | | |
| (11%) | | | | |
| Propylene | 99.66 | 99.51 | 99.65 | 99.58 |
| glycol (10%) | | | | |
| Ethanol (12.5%)/ | | | | |
| Benzyl alcohol | | | | |
| (12.5%) | | | | |

TABLE 5
HPLC Results for Faslodex formulation after 1 month stability trial

| | % Fulvestrant | % Fulvestrant | % Fulvestrant | % Fulvestrant |
|---|---|---|---|---|
| Formulation | at start date | at 40° C. | at Room Temperature | at 2-8° C. |
| Faslodex | 99.16 | 99.12 | 99.45 | 99.32 |

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A formulation comprising:
an active compound selected from:
(i) fulvestrant or a pharmaceutically acceptable salt thereof; and
(ii) a pharmaceutically acceptable derivative of fulvestrant or a pharmaceutically acceptable salt thereof;
at least one pharmaceutically acceptable alcohol;
at least one of propylene glycol or a pharmaceutically acceptable polyethylene glycol; and
a castor oil carrier.

2. A formulation according to claim 1, wherein the propylene glycol or polyethylene glycol is in the range 10% w/w to 20% w/w of the formulation.

3. A formulation according to claim 2, wherein the propylene glycol or polyethylene glycol is 15% w/w of the formulation.

4. A formulation according to claim 1, wherein the polyethylene glycol is selected from the group consisting of PEG-200, PEG-300, PEG-400 and PEG-600.

5. A formulation according to claim 4, wherein the polyethylene glycol is PEG-300.

6. A formulation according to claim 1, wherein the pharmaceutically acceptable alcohol is selected from the group consisting of ethanol, benzyl alcohol or a mixture thereof.

7. A formulation according to claim 6, wherein the pharmaceutically acceptable alcohol is ethanol.

8. A formulation according to claim 7, wherein the ethanol is in the range 8% w/w to 15% w/w of the formulation.

9. A formulation according to claim 7, wherein the ethanol is 10% w/w of the formulation.

10. A formulation according to claim 9, wherein the pharmaceutically acceptable alcohol is benzyl alcohol.

11. A formulation according to claim 10, wherein the benzyl alcohol is in the range 5% w/w to 15% w/w of the formulation.

12. A formulation according to claim 10, wherein the benzyl alcohol is 10% w/w of the formulation.

13. A formulation according to claim 6, wherein the pharmaceutically acceptable alcohol is a mixture of ethanol and benzyl alcohol.

14. A formulation according to claim 6, wherein the mixture is in the range 15% w/w to 25% w/w of the formulation.

15. A formulation according to claim 6 wherein the mixture is 20% w/w of the formulation.

16. A formulation according to claim 1, wherein the effective amount of the active compound is in the range 2% w/w to 10% w/w of the formulation.

17. A formulation according to claim 1, wherein the effective amount of the active compound is 5% w/w of the formulation.

18. A formulation comprising:
(a) around 5% w/w of an active compound selected from:
(i) fulvestrant or a pharmaceutically acceptable salt thereof; and
(ii) a pharmaceutically acceptable derivative of fulvestrant or a pharmaceutically acceptable salt thereof;
(b) 10% w/w of ethanol;
(c) 10% w/w of benzyl alcohol;
(d) 15% w/w of PEG 300; and
(e) castor oil to make up the required weight.
